(19) United States
(12) Patent Application Publication
(10) Pub. No.: US 2026/0351334 A1
(43) Pub. Date: Nov. 5, 2026

(54) METHOD AND SYSTEM FOR PROBABILISTIC TYPING OF MICROBIAL STRAINS

(71) Applicant: BIOMÉRIEUX, Marcy L'etoile (FR)

(72) Inventors: Hannes POUSEELE, Oostende (BE); Meylan TETREAU, Meylan (FR); Joao Andre CARRIÇ0, Amadora (PT)

(73) Assignee: BIOMÉRIEUX, Marcy L'etoile (FR)

(21) Appl. No.: 19/487,298

(22) PCT Filed: May 30, 2024
(86) PCT No.: PCT/EP2024/064927

§ 371 Date: Nov. 24, 2025

(30) Foreign Application Priority Data
May 31, 2023 (EP) 23176321.0

(51) Int. Cl. G16B 10/00; C12Q 1/6806; C12Q 1/689; G16B 20/20

(52) U.S. Cl. CPC G16B 10/00; C12Q 1/6806; C12Q 1/689; G16B 20/20; C12Q 2600/156

(57) ABSTRACT

A microbiological typing method, which includes providing a database of genetic profiles of microbial strains, a phylogenetic tree, assignments of the genetic profiles to positions in the phylogenetic tree, and frequencies of variation of the profiles in the tree. The typing of a microorganism includes measuring the genetic profile of the microorganism, determining probabilities of variation of the genetic profiles belonging to the groups with respect to the measured genetic profile, the probabilities of variation being calculated as a function of the frequencies of variation at a predefined level of the phylogenetic tree and of the belonging of the microorganism to one of the microbial groups if an identification performance criterion is reached for the group, the criterion being reached if at least one probability of variation for the group is greater than a first predefined threshold.

### FIELD OF THE INVENTION

The invention relates to the field of genomic typing of microorganisms, namely the characterization of bacterial strains at a taxonomic level below the species level based on their genomes. The invention is particularly applicable in the epidemiological monitoring of bacterial strains, the monitoring of contamination by bacterial strains, and the search for the root causes of bacterial contamination and/or infection, whether of industrial, environmental, veterinary, clinical, or other origin.

### PRIOR ART

Genomic typing of microorganisms, for example bacteria, combines all the techniques for comparing the genomes of bacterial strains in order to characterize them at levels below the species level. Typing makes it possible, notably, to determine the degree of similarity between two bacterial strains or to classify strains into subdivisions of the species level, for example clonal groups or groups defined by phenotypic features of bacteria such as their serotypes or their susceptibility to antibiotics.

Typing techniques may be classified into two categories, the first based on the analysis of a very small targeted portion of the bacterial genome, such as “16S rRNA” MLST (Multi-Locus Sequence Typing) typing, and the second based on the comparison of genomes obtained by whole genome sequencing (WGS), for example cg-MLST (core genome MLST) typing. While the latter have the highest level of accuracy, whole genome sequencing has many drawbacks, including time, cost, and the need to perform complex bioinformatics treatments. In contrast, when the first techniques use a very limited number of markers, they allow the use of polymerase chain reaction (PCR) amplification platforms for their detection, which are much faster and less expensive.

Regardless of the technique used, the genomic typing of the prior art is based on the search for and use of conserved portions of the genome to characterize microorganisms. For example, MLST typing is based on housekeeping genes that are highly stable over time; similarly, cg-MLST typing directly targets all of the core genome (or basic genome: these terms are used interchangeably). With this type of approach, the production of high accuracy requires a very large number of markers. By way of example, in order to detect clonal groups of the species P. aeruginosa with an accuracy of over 90%, cg-MLST typing requires several thousand markers, consequently making the use of conventional PCR platforms irrelevant or even impossible.

To summarize, a user wishing to perform molecular typing, for example for the purpose of monitoring microbial contamination, must choose between accuracy and speed.

### DESCRIPTION OF THE INVENTION

The aim of the present invention is to propose a molecular typing method using a limited number of highly accurate genetic markers.

To this end, the invention relates to an in vitro process for typing a microorganism contained in a sample, which involves:
a. providing computer storage means comprising:
   a database of genetic profiles of microbial strains belonging to the microbial species of said microorganism, said profiles consisting of the presence or absence of a predefined set of genetic markers;
   a phylogenetic tree of the species of said microorganism, assignments of the genetic profiles in the database to positions in the phylogenetic tree, frequencies of variation of the genetic profiles of the microbial strains in the database for predefined levels of the phylogenetic tree;
   for at least part of the genetic profiles in the database, the membership of said genetic profiles to distinct microbial groups of the microbial species, said distinct groups being absent from the phylogenetic tree;
b. the measurement of the genetic profile of the microorganism;
c. the computer-mediated determination of:
   probabilities of variation of the genetic profiles belonging to said groups relative to the measured genetic profile, said probabilities of variation being calculated as a function of the frequencies of variation at a predefined level of the phylogenetic tree;
   the microorganism's membership in one of the microbial groups if an identification performance criterion is met for said group, said criterion being met if at least one probability of variation for said group is greater than a first predefined threshold.

According to one embodiment:
the phylogenetic tree is constructed from a core genome of the microorganism species;
the predefined set of genetic markers is chosen from the accessory genome of the microorganism species.

According to one embodiment, the number of markers in the genetic profiles is less than 50, and preferably between 16 and 30.

According to one embodiment:
the phylogenetic tree is constructed by the application of clustering based on genetic distances of a core genome of the microorganism species;
the genetic distances decrease from the root to the leaves of the tree, so as to define a level in the tree comprising between 150 and 350 differences in the core genome.

In particular:
the groups of microorganisms correspond to microbial serotypes of the microorganism species;
the phylogenetic tree comprises at least 10 levels, preferentially at least 15 levels;
the probabilities of variation of the genetic profiles belonging to said groups relative to the measured genetic profile are calculated as a function of the frequencies of variation of a level of the phylogenetic tree between 7 and 15, preferentially between 10 and 15, more preferentially between 11 and 13.

According to one embodiment, the genetic profile markers are selected so as to minimize entropy or impurity criteria at a level of the phylogenetic tree comprising between 150 and 350 differences in the core genome.

According to one embodiment, the identification performance criterion also includes the difference between the probability of variation for said group and the probabilities of variation for the other groups, the identification performance criterion being met if said difference is greater than a second predefined threshold. In particular, the identification performance indicator comprises a correct degree of assignment and/or a raw accuracy and/or a balanced accuracy.

According to one embodiment, the process involves a search for root causes in the event of contamination of a food product.

According to one embodiment, the genetic profile of the microorganism is measured by amplifying target genetic sequences without performing complete sequencing, in particular via a polymerization chain reaction.

A subject of the invention is also a system for the in vitro typing of a microorganism contained in a sample, comprising:
a. computer storage means comprising:
   a database of genetic profiles of microbial strains belonging to the microbial species of said microorganism, said profiles consisting of the presence or absence of a predefined set of genetic markers;
   a phylogenetic tree of the species of said microorganism, assignments of the genetic profiles in the database to positions in the phylogenetic tree, frequencies of variation of the genetic profiles of the microbial strains in the database for predefined levels of the phylogenetic tree;
b. means for collecting and storing a measurement of the genetic profile of the microorganism;
c. computer-mediated means for determining:
   probabilities of variation of the genetic profiles belonging to said groups relative to the measured genetic profile, said probabilities of variation being calculated as a function of the frequencies of variation at a predefined level of the phylogenetic tree;
   the microorganism's membership in one of the microbial groups if an identification performance criterion is met for said group, said criterion being met if at least one probability of variation for said group is greater than a first predefined threshold.

According to one embodiment, the system is configured to perform a process of the abovementioned type.

A subject of the invention is also a computer program product recorded on a computer-readable support comprising instructions for executing step c. of a process of the abovementioned type.

A subject of the invention is also a composition for the in vitro typing of microorganisms contained in a sample by amplification of target nucleotide sequences, in particular by a polymerization chain reaction, said composition comprising a set of oligonucleotides for the amplification of at least the set of genetic markers obtained according to the process of the abovementioned type.

A subject of the invention is also a process for preparing a biological sample comprising or liable to comprise a bacterial strain, for typing said strain by a polymerization chain reaction, which involves placing said sample in contact with a composition of the abovementioned type.

According to one embodiment, the process involves lyzing the sample obtained from said placing in contact so as to release genetic material from the bacteria of the bacterial strain.

A subject of the invention is also a process for preparing a sample which may comprise a microorganism, said process involving
the extraction of the genetic material included in the sample;
mixing of said extracted genetic material with a composition comprising a set of primers for amplification, in particular by polymerase chain reaction, of a set of genetic markers obtained according to a process of the abovementioned type.

### BRIEF DESCRIPTION OF THE FIGURES

The invention will be better understood on reading the description that follows, which is given solely by way of example and is produced in relation with the accompanying drawings, in which:

FIG. 1 is an overview of a process according to the invention;

FIG. 2 is a flowchart detailing a learning phase of a phylogenetic typing according to the invention;

FIG. 3 is a graph illustrating a decreasing genomic distance for the construction of a phylogenetic tree;

FIG. 4 is a schematic view of a binary phylogenetic tree;

FIG. 5 is a scheme illustrating the calculation of the frequencies of variation of a genetic profile according to the invention for a taxon of the phylogenetic tree;

FIG. 6 is a flowchart illustrating the production of a PCR kit to be used for the typing according to the invention;

FIG. 7 is a flowchart detailing a phylogenetic type prediction phase according to the invention;

FIG. 8 is a flowchart illustrating the calculation of thresholds used during the prediction phase;

FIG. 9 is a flowchart illustrating a learning phase for serotyping according to the invention;

FIG. 10 is a flowchart illustrating a serotype prediction phase according to the invention;

FIG. 11 is a flowchart illustrating the calculation of thresholds used during the serotype prediction phase;

FIG. 12 is a graph of the decrease in Shannon entropy during the selection of genetic markers constituting a genetic profile for Listeria typing;

FIG. 13 is a set of graphs of the frequencies of variation of genetic profiles in the phylogenetic tree of Listeria;

FIG. 14 is a graph of the correct serotypic degree of assignment for Listeria as a function of balanced accuracy;

FIG. 15 is a set of graphs of the correct phylogenetic degree of assignment for Listeria as a function of an average accuracy;

FIG. 16 is a graph of the decrease in Shannon entropy during the selection of genetic markers constituting a genetic profile for Salmonella typing;

FIG. 17 is a set of graphs of the frequencies of variation of genetic profiles in the phylogenetic tree of Salmonella;

FIG. 18 is a graph of the correct serotypic degree of assignment for Salmonella as a function of balanced accuracy;

FIG. 19 is a set of graphs of the correct phylogenetic degree of assignment for Salmonella as a function of average accuracy; and

FIGS. 20 to 22 illustrate computer architectures for performing the typing according to the invention.

### DETAILED DESCRIPTION OF THE INVENTION

### A. Definitions

In the present document, the following definitions apply:
Biological sample: denotes any sample containing or liable to contain one or more microorganisms;
Core genome (or “basic genome”, denoted “Gb”) of a microbial species: refers to parts of the genome (genes or groups of genes) shared by all the microbial strains belonging to the one or more microbial species under consideration. For the purposes of the invention, the core genome may evolve if the set of strains from which it is produced evolves. Preferably, the core genome is constructed from at least 100 different microbial strains, and preferably from at least 1000 strains. It will be noted that the genomic portions of microbial strains belonging to the core genome may differ from each other. For example, a particular gene may belong to the core genome and have several different alleles. In the text hereinbelow, the term “core genome” when applied to a microbial strain refers to the portion of the strain's genome that belongs to the core genome of the species.
Accessory genome of a microbial species (denoted “Ga”): denotes parts of the genome (genes or groups of genes) of a set of bacterial strains that do not belong to the core genome of said set. These parts of the genome are thus absent from the genome of at least one of the bacterial strains in the set of bacterial strains under consideration. For the purposes of the invention, the accessory genome may evolve if the set of strains from which it is produced evolves. Preferably, the accessory genome is constructed from at least 100 different microbial strains, and preferably from at least 1000 strains.
Genetic marker: denotes a gene or nucleotide sequence at a specific position in the genome (also called a “locus”), a nucleotide sequence that may amount to a single nucleotide, for instance two alleles differing by a single nucleotide, or a single nucleotide polymorphism in the context of a mutation (or SNP for “single nucleotide polymorphism”);
Microorganism: denotes all microscopic organisms, notably including bacteria, yeasts, and fungi.
Phylogenetic tree of a microbial species: denotes a tree representing evolutionary relationships among a set of microbial strains, determined as a function of their similarities and differences in their core genomes. For the purposes of the invention, a phylogenetic tree can be enriched if the set of strains from which it is produced evolves. Preferably, a phylogenetic tree is constructed from at least 100 different microbial strains, and preferably from at least 1000 strains. By convention, the root of a phylogenetic tree is denoted as the first level of the tree and the leaves of the tree as the last levels. A level that is “higher” than another one thus corresponds to a level that is closer to the root.

### B. Embodiment of the Invention

Disclosed hereinbelow is a probabilistic typing method according to the invention applied to the monitoring of contamination with bacterial strains of a species within an organization, in particular in premises for producing food intended for human consumption, and also to the detection of their serotypes.

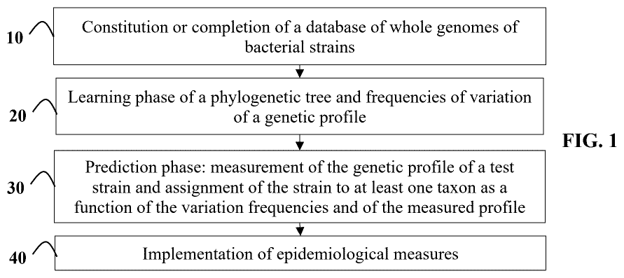

With reference to FIG. 1, the typing according to the invention comprises the following main phases:
a phase 10 of establishing a database consisting of complete genomes of strains belonging to the bacterial species;
a phase 20 known as “learning” or “training”, directed toward generating a phylogenetic tree as a function of the genomes in the database, choosing an appropriate genetic profile and the frequencies of variation of said genetic profile along the tree;
a “prediction” phase 30, performing the typing of an unknown bacterial strain as a function of the tree and of the frequencies produced in phase 20; and
phases that are optionally supplemented with an “epidemiological” phase 40 during which corrective and/or prophylactic measures are taken within the organization as a function of the result of the typing of the unknown strain.

Preferably, the database of complete genomes is as representative as possible of the genetic diversity of the species. It is advantageously supplemented on an ongoing basis by adding complete genomes so as to target this diversity if the initial version is incomplete. In this case, the typing according to the invention is performed when said database comprises at least 100 genomes of the species, preferably at least 1000 genomes, and even more preferably several thousand genomes.

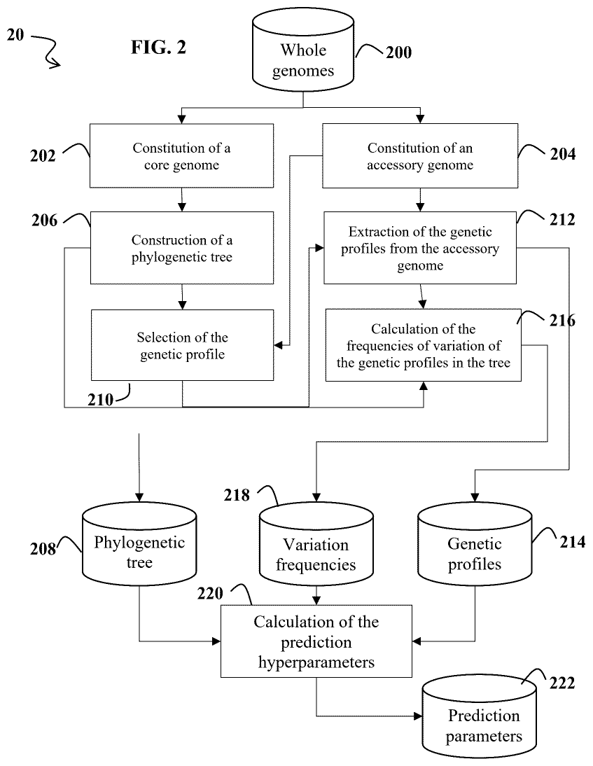

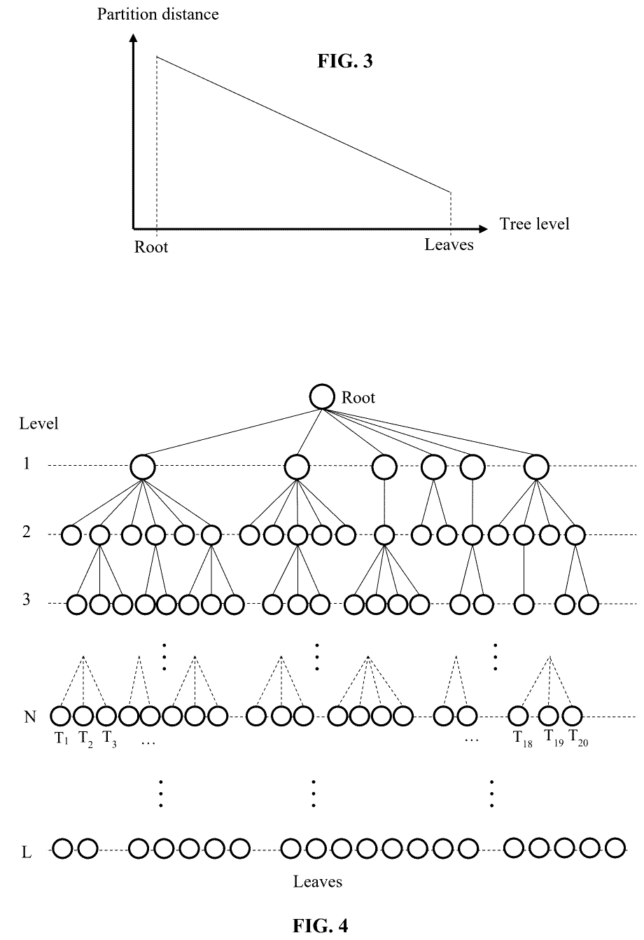

With reference to FIGS. 2, 3 and 4, the training phase 20 begins with the production of a core genome in 202 and an accessory genome in 204 from the genomes in database 200. In a first variant, the genomes comprise annotations categorizing the genes as belonging to one or other of these genomes. In a second variant, these are produced ad hoc from the database, with genes having an allele frequency above a threshold, for example 95%, belonging to the core genome, or else to the accessory genome. In a third variant, the core and accessory genomes are produced from the database by comparison with a reference genome of the species stored in the database, with genes having an allele frequency relative to the reference genome greater than a threshold, for example 95%, belonging to the core genome, or else to the accessory genome. In a fourth variant, the database is already established for the species, as is a version of its core and accessory genomes.

The typing according to the invention continues in 206 with the generation of a phylogenetic tree as a function of the core genome. Several techniques for constructing such a tree are possible, as described, for instance, in the documents “Phylogenetics Algorithms and Application” (Hu et al., Ambient Communications and Computer Systems, 2019), “A Review: Phylogeny Construction Methods” (Shaktawat et al., International Journal of Emerging Science and Engineering, 2019) or “Essential Bioinformatics” (chapter 11 by Jin Xiong, Cambridge University, 2011).

In a variant of the invention, the tree is constructed from cg-MLST (core-genome multi-locus sequencing typing) coding of the core genomes. These are translated into cg-MLST profiles using, for example, the BioNumerics software (bioMerieux, Marcy l′Etoile, France), SeqSphere+ (Ridom, Münster, Germany), BIGSdb (accessible at https://github.com/kjolley/BIGSdb and described in the document by Schürch, A. et al. “Whole genome sequencing options for bacterial strain typing and epidemiologic analysis based on single nucleotide polymorphism versus gene-by-gene-based approaches”. Clin. Microbiol. Infect., 2018), or chewBBACA (accessible at https://github.com/B-UMMI/chewBBACA and described in the document by Silva M et al., “chewBBACA: a complete suite for gene-by-gene scheme creation and strain identification.” Microb. Genom., 2018). The advantage of cg-MLST profiles is that they are sequences of fixed lengths, consisting of a few hundred components, and aligned relative to each other, allowing the direct use of tree construction methods.

This tree is advantageously generated by applying a series of groupings using the single-linkage clustering method, and is thus based on genetic distances between the core genomes, calculated using the normalized Hamming distance, for example. The subdivision of a taxon in the tree then consists in combining together in the same subgroup the core genomes that differ from each other by a distance less than a predefined threshold. For example, software such as HierCC (accessible at https://github.com/zheminzhou/pHierCC and described in the document “HierCC: a multi-level clustering scheme for population assignments based on core genome MLST” by Zhou et al., Bioinformatics, 2021) and Pathogenwatch from the Center for Genomic Pathogen Surveillance (accessible at https://pathogen.watch/) may be used. Once the tree has been constructed, it is stored in a database 208.

In a particularly preferred variant of the invention, the number of levels in the tree and the clustering distances are chosen so as to produce at least one level in the tree such that the diversity of the core genome at that level corresponds to or is close to the clonal diversity observed in the species, so that the taxa at that level allow different clonal complexes to be distinguished among the bacterial strains corresponding to the complete genomes in the database. For example, for Salmonella and Listeria, the level has between 150 and 350 differences in the core genome of the strains, preferably between 200 and 300 differences. In particular, the clustering distance decreases from the root to the leaves, for example linearly.

The learning phase 20 continues with the selection, in 210, of a type of genetic profile in the accessory genome, i.e. a set of genetic markers from the accessory genome of the species whose absence and presence are measured in a strain to be tested during the prediction phase 30. The choice of profile consists in choosing a level in the tree, and preferably one of the levels representing the clonal diversity, and then choosing a set of markers having a minimal entropy or degree of impurity at that level. The total number NM of markers selected to construct the genetic profile is advantageously selected as a function of the particular PCR platform used to perform the typing. For example, for the Applicant's GENE-UP platform, this number NM is equal to 23 markers for Salmonella and 16 markers for Listeria monocytogenes.

In practice, the set of markers is chosen so as to minimize an entropy criterion (for example, Rényi entropy, Hartley entropy, Shannon entropy, collision entropy, or min entropy) or a Gini impurity criterion, using a greedy algorithm.

For illustrative purposes, and with reference to the phylogenetic tree in FIG. 4, the selection of a set of markers to minimize the Shannon entropy criterion of level N consisting of Q_{N}=20 taxa T₁, T₂, . . . , T₂₀ consists in choosing a first marker M₁ from the accessory genome that minimizes the entropy criterion:

$${E}_{1} = - {\sum }_{i = 1}^{{Q}_{N}} p ( i ) {log}_{2} p ( i )$$

in which

$$p ( i ) = \frac{{n}_{i}}{NG}$$

where NG is the total number of core genomes used to construct the phylogenetic tree, and nᵢ the number of times the marker is present in the accessory genomes in the i^{th} taxon Tᵢ. Once marker M₁ has been selected, this procedure is repeated to select the second marker M₂ in the accessory genome removed from marker M₁. Thus, the j^{th} marker Mⱼ is the marker such that

$${M}_{j} = arg {min}_{{M}_{j} ∈ Ga , {M}_{j} ∉ \{ {M}_{1} , {M}_{2} , … , {M}_{j - 1} \}} - {\sum }_{i = 1}^{{Q}_{N}} p ( i ) {log}_{2} p ( i )$$

$$p ( i ) = \frac{{n}_{i}}{N G} \qquad ( i )$$

where NG is the total number of core genomes used to construct the phylogenetic tree and nᵢ is the number of times that the marker Mⱼ is present in the accessory genomes in the i^{th} taxon Tᵢ.

As a variant, stepwise marker selection is performed by minimizing the conditional entropy of level N according to the formula:

$${M}_{j} = arg {min}_{{M}_{j} ∈ G a , {M}_{j} ∉ \{ {M}_{1} , {M}_{2} , … , {M}_{j - 1} \}} - {\sum }_{i = 1}^{{Q}_{N}} {\sum }_{l = 1}^{{2}^{j}} p ( i ) p ( {M}_{l} \vert i ) {log}_{2} p ( i ) p ( {M}_{l} \vert i )$$

where:
in which

$$p ( i ) = \frac{{n}_{i}}{N G}$$

where NG is the total number of core genomes used to construct the phylogenetic tree, and nᵢ the number of times the marker is present in the accessory genomes in the i^{th} taxon Tᵢ.
and

$$p ( {M}_{l} \vert i ) = \frac{{n}_{l}}{{N}_{i}}$$

with nₗ number of times that the 1^{th} value of the marker set {M₁, M₂, . . . , Mⱼ₋₁, Mⱼ}, among the 2^{j} possible values of said set, is present in the i^{th} taxon Tᵢ and Nᵢ the number of core genomes included in the taxon Tᵢ.

The inventors have observed that the typing according to the invention requires not more than 50 markers, preferably fewer than 30 markers, more particularly a number between 16 and 30 markers, to obtain high typing performance.

The learning phase 20 continues in 212 with the extraction of accessory genomes from the genetic profiles formed of the selected markers, which are stored in a database 214. The genetic profile of an accessory genome is therefore a vector P of {0.1}^{N}^{_{M}}, where the j^{th} component P(j) of the vector P corresponds to the value of the marker Mⱼ, this value being equal to 0 when the marker M is absent and 1 when it is present.

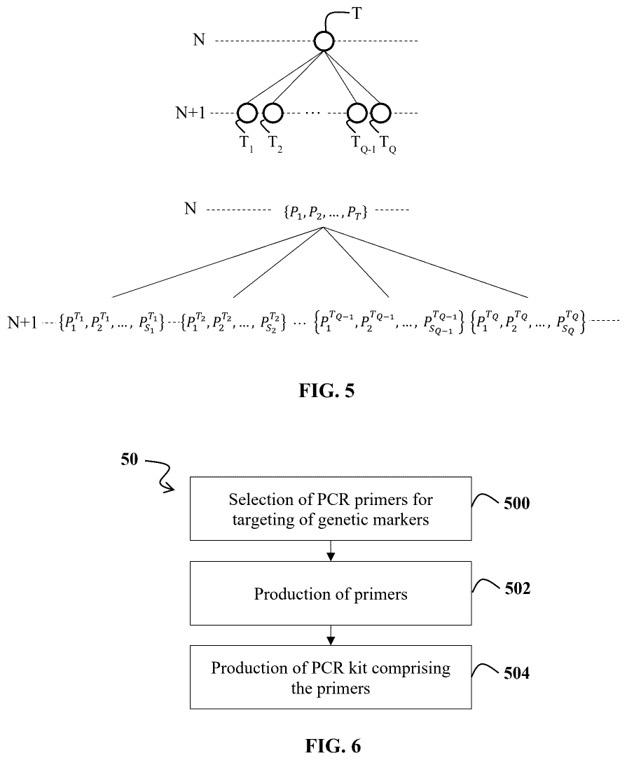

Once the profile database 214 is formed, the frequencies of variation of the profiles along the tree are calculated in 216. With reference to to FIG. 5, for each taxon T of the tree that is partitioned into subdivisions T₁, T₂, . . . , T_{Q}, there is a set of genetic profiles {P₁, P₂, . . . , P₇} associated with the taxon T partitioned into subsets of profiles

$$\{ {P}_{1}^{{T}_{1}} , {P}_{2}^{{T}_{1}} , … , {P}_{{S}_{1}}^{{T}_{1}} \} , \{ {P}_{1}^{{T}_{2}} , {P}_{2}^{{T}_{2}} , … , {P}_{{S}_{2}}^{{T}_{2}} \} , … , \{ {P}_{1}^{{T}_{Q}} , {P}_{2}^{{T}_{Q}} , … , {P}_{{S}_{Q}}^{{T}_{Q}} \}$$

respectively associated with the subtaxa T₁, T₂, . . . , T_{Q}. For each marker Mⱼ of the genetic profile, a variation frequency P(Mj|T) of this marker for taxon T is calculated as equal to the number, divided by Q, of subtaxa T₁, T₂, . . . , T_{Q} for which the marker Mⱼ takes both the value 0 and the value 1, i.e. a frequency according to the relationship:

$$P ( {M}_{j} \vert T ) = \frac{card ( \{ {T}_{q} ∖ ∃ ( v , w ) , u \neq W , {P}_{u}^{{T}_{q}} ( j ) = 0 \& {P}_{w}^{{T}_{q}} ( j ) = 1 \} )}{Q}$$

in which card(E) denotes the cardinality function of a set E. The calculation of frequencies thus produces, for each subdivided taxon T, a frequency vector P(.|T) of [0.1]^{N}^{_{M}} such that P(.|T)(j)=P(Mⱼ|T). Once calculated, the frequency vectors are stored in a database 218.

The learning phase 20 terminates with the calculation, in 220, of a grid of hyperparameters used during the prediction phase 30, which calculation will be detailed hereinbelow once prediction 30 has been described, the parameters being stored in a database 222.

In summary, the learning phase 20 provides a phylogenetic tree in which each complete genome and its genetic profile, extracted from its accessory genome, is positioned in the tree and each taxon in the tree, excluding the leaves, is associated with a frequency of variation of each genetic marker in the profile.

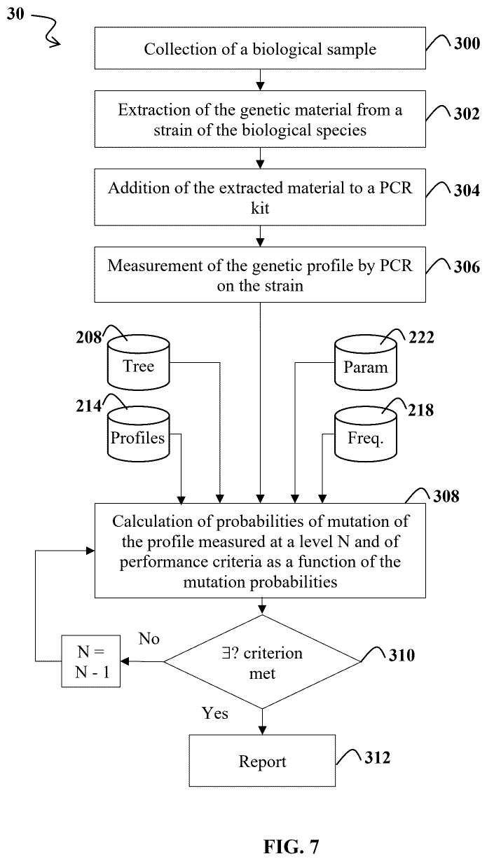

With reference to FIG. 6, the invention includes the design and production 50 of a composition, or “kit”, for performing amplification aimed at detecting the selected markers, comprising:
the selection, in 500, of amplification primers for the amplification of the selected genetic markers:
the production of said primers in 502 and, if necessary, the production of detection probes so as to detect the presence of a marker in at least one of its variants, and preferentially in the maximum number of its variants;
the production, in 504, of a buffer solution comprising the selected primers, the detection probes if necessary, dNTPs (deoxyribonucleotide triphosphates) to afford the energy and nucleotide bases necessary for the production of amplicons, an amplification enzyme and salts such as Mg²⁺ ions or NaCl to allow the enzyme to function properly, at appropriate concentrations. Dyes may be added to this solution.
the production of a lysis buffer comprising beads, for example magnetic or ceramic beads, and optionally a dye.

The kits produced are then used during prediction phase 30. With reference to FIG. 7, this phase begins with the collection 300 of a biological sample from the organization, for example a food product production facility. During a subsequent step 302, the sample is prepared so as to detect a given pathogen using methods known in the prior art, such as culturing on a plate, VIDAS®-type immunological detection, or molecular biology, for example using the GENE-UP® platform. Following detection of said pathogen, a confirmation step may take place.

At the same time, if the colony is identified as belonging to the species, the prediction phase 30 continues in 302 using isolated colonies or an enrichment broth for the purpose of characterization by PCR amplification. In particular, the sample undergoes a series of preliminary steps, such as lysis to release the DNA from the bacteria constituting it, and optionally extraction of the DNA from the lysis using beads, for instance magnetic or ceramic beads. Once the DNA of the bacterial strain has been released, it is added, in 304, to the PCR kit designed to target at least the genetic markers selected during the learning step 20. Finally, PCR is performed in 306 so as to measure the genetic profile of the strain, for example using the Applicant's GENE-UP® platform.

The measured profile, denoted Pₘₑₛ, is then analyzed in 308 so as to position the bacterial strain in the phylogenetic tree. Starting from a level N of the tree, for example the level directly above the leaf level, the analysis 308 comprises the following steps:
a. for each taxon at level N, denoted T_{i,N} (i^{th} taxon of the Nth level):
   a1. for each stored profile, denoted P_{obs}, in database 214 that is assigned to taxon
      Tᵢ,N, the calculation of a probability Pₘᵤₜ(Pₘₑₛ,P_{obs}) that the measured profile
      Pₘₑₛ is a variation of the stored profile P_{obs} according to the relationship:

$${P}_{m u t} ( {P}_{mes} , {P}_{o b s} ) = {\prod }_{{j = 1 , … , {N}_{m}}_{{P}_{meas} ( j ) \neq {P}_{obs} ( j )}} {P}_{m u t} ( {M}_{j} \vert {T}_{i , N} )$$

It will thus be noted that if the measured profile Pₘₑₛ is identical to the profile P_{obs}, then this probability is equal to 1 (no variation) and reduces rapidly if the profile Pₘₑₛ deviates from the profile P_{obs}, all the more so when the frequencies Pₘᵤₜ(Mⱼ|Tᵢ,N) are low.
a2. selection of the maximum

$${P}_{m u t}^{max} ( i , N )$$

of the probabilities Pₘᵤₜ(Pₘₑₛ,P_{obs}) calculated for the taxon T_{i,N}.
b. Assignment, in 310, of the measured profile Pₘₑₛ to one of the taxa T_{j,N} of level N such that:

$$\{ \begin{matrix}{T}_{j , N} = arg {max}_{i} {P}_{m u t}^{max} ( i , N ) \\ {P}_{m ut}^{max} ( j , N ) \geq {S}_{1} \\ ∀ i \neq j , {P}_{m u t}^{max} ( j , N ) - {P}_{m u t}^{max} ( i , N ) \geq {S}_{2}\end{matrix}$$

in which S₁ and S₂ are predetermined nonzero positive thresholds;
c. If no taxon of level N satisfies the criterion in step b, move to the higher level N−1 of the tree and perform steps a and b for this new level.

Once the profile Pₘₑₛ has been assigned to a level of the phylogenetic tree and a taxon of that level, prediction phase 30 delivers a report to the user, for example in the form of a display on a screen and/or files stored on a computer and/or a message (e.g. email) to the user. If two taxa have identical results on conclusion of step b, both taxa are included or an inconclusive typing is noted in the report.

The position of the new bacterial strain tested in the phylogenetic tree allows it to be typed relative to the bacterial strains corresponding to the complete genomes in database 200. Besides this type in reference to strains previously observed and capitalized in the various databases, the typing according to the invention allows the relative typing of two bacterial strains if these two strains are assigned to the same taxon. Furthermore, the invention makes it possible to define the lineage of a strain relative to those in the databases or relative to another tested strain. Specifically, the relative position of two strains in the tree allows their relative evolution to be characterized.

A path through the phylogenetic tree from the leaves to the root has been described: as long as the assignment relationships are not verified, the tree is traced upward. As a variant, the phylogenetic tree is traced from the root to the leaves: as long as the assignment relationship is verified, the tree is traced downward.

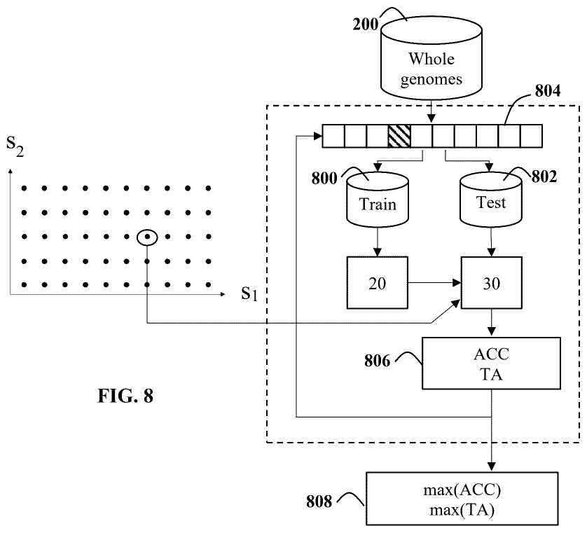

The values of the thresholds S₁ and S₂ are hyperparameters determined during step 220 of learning phase 30, preferably by means of cross-validation. With reference to FIG. 8, a grid of values for the thresholds S₁ and S₂ is first determined, and for each pair of thresholds in the grid, a determination of the correct degree of assignment and the average accuracy is performed by:
partitioning the complete genome database 200 into a training database 800 and a test database 802 according to a cross-validation strategy 804, for example a strategy called “ten-folds cross validation”;
performing the learning phase 20 on the training database 802;

performing the prediction phase 30 for each genetic profile from the test base 802 and calculating in 806 the average accuracy ACC and the correct degree of assignment TA of the genetic profiles from the database 802 in the phylogenetic tree;
the selection, in 808, of the maximum value of the average accuracies and correct degrees of assignment among the folds of the cross-validation strategy. These two values therefore correspond respectively to the average accuracy and the correct degree of assignment of the phylogenetic prediction phase 30 for the thresholds S₁ and S₂. The pairs of thresholds having the best accuracy and degree are selected for the phylogenetic typing according to the invention. As a variant, the user can choose the accuracy and/or degree of assignment they desire, and the phylogenetic typing selects the pair of thresholds whose accuracy and degree are closest to those chosen by the user.

In a preferred manner, the number of levels in the phylogenetic tree and the law of distance decay of the clustering, for example affine, are also hyperparameters, the process described in relation to FIG. 8 thus being supplemented with the selection of a grid for these two parameters, and the path of said grid in conjunction with the path of the grid of thresholds S₁ and S₂.

A preferred variant of the invention is now described, consisting in predicting the membership of a bacterial strain in a particular group of the bacterial species as a function of its measured genetic profile. In the example shown, these groups are serotypes, but this variant applies to any explicit subdivision at a level of the bacterial species. It will be noted that this subdivision of the species does not correspond to the first level of the phylogenetic tree and is defined independently thereof.

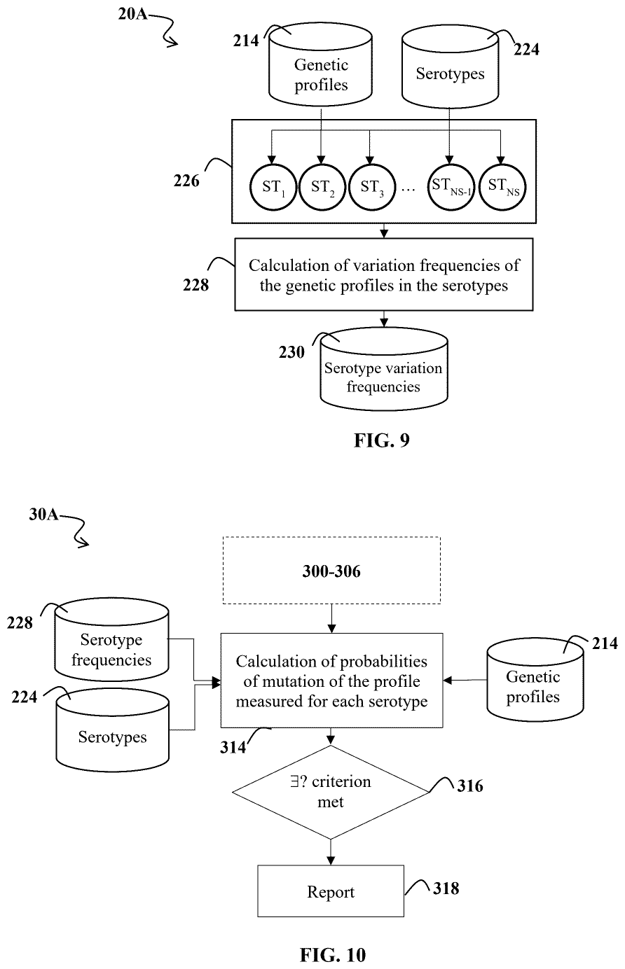

With reference to FIG. 9, the learning phase 20 described above in relation to FIG. 2 is supplemented with serotype learning 20A comprising:
a. the collection of serotypes from all or some of the bacterial strains corresponding to the complete genomes, the serotypes being stored in a database 224;
b. the distribution, in 226, of the genetic profiles observed in database 214 of the serotyped strains among the NS different serotypes ST1, ST2, . . . . ST_{NS}. The genetic profiles associated with a serotype are referred to hereinbelow as “serotyped”;
c. the calculation, 228, of the frequencies of variation of the serotyped genetic profiles. Notably, for each marker Mⱼ, its frequency of variation P(Mⱼ|STᵢ) in a serotype STi is equal to the number of times that this marker takes the value 1 out of the total number of genetic profiles assigned to the serotype STi;
d. the storage in a database 230 of the frequencies of variation P(Mⱼ|STᵢ) of the serotyped genetic profiles in the different serotypes.

If the elements of learning phase 20 described in relation to FIG. 2 remain unchanged, notably meaning that the type of genetic profiles used is determined as a function of the phylogenetic tree and not as a function of serotype subdivision, prediction phase 30 may take two forms. In the first, phase 30 already described is supplemented with a prediction of the serotype of a new strain to be tested, in parallel with steps 308-312 in FIG. 2. In the second form, only said prediction is performed and replaces steps 308-312.

With reference to FIG. 10, prediction 30A of the serotype of a new bacterial strain follows steps 300-306 measuring the genetic profile of a bacterial strain of the species contained in a biological sample, and comprises the following steps:
a. for each serotype, denoted STᵢ:
   a1. for each serotyped genetic profile, denoted P_{ST}, of the group STᵢ of database 228, the calculation of a probability Pₘᵤₜ(Pₘₑₛ,P_{ST}) that the measured profile Pₘₑₛ is a variation of the stored profile Psr according to the relationship:

$${P}_{m u t} ( {P}_{mes} , {P}_{S T} ) = {\prod }_{{j = 1 , … , {N}_{m}}_{{P}_{meas} ( j ) \neq {P}_{S T} ( j )}} {P}_{m u t} ( {M}_{j} \vert S {T}_{i} )$$

a2. selection of the maximum

$${P}_{m u t}^{max} ( S {T}_{i} )$$

of the probabilities Pₘᵤₜ(Pₘₑₛ,P_{obs}) calculated for the serotype STᵢ;
b. Assignment of the measured profile Pₘₑₛ to one of the serotypes STⱼ such that:

$$\{ \begin{matrix}S {T}_{j} = arg {max}_{i} {P}_{m u t}^{max} ( S {T}_{i} ) \\ {P}_{m u t}^{max} ( S {T}_{j} ) \geq {S}_{3} \\ ∀ i \neq j , {P}_{m u t}^{max} ( S {T}_{j} ) - {P}_{m u t}^{max} ( {ST}_{i} ) \geq {S}_{4}\end{matrix}$$

in which S₃ and S₄ are predetermined nonzero positive thresholds;

c. Production and delivery, in 318, of a report on the serotype detected for the bacterial strain which has undergone the serotype typing.

In a variant of the serotyping 30A, if the second condition

$${P}_{m u t}^{max} ( S {T}_{j} ) - {P}_{m u t}^{max} ( S {T}_{i} ) \geq {S}_{4}$$

is not satisfied, the serotyping returns the first two or three serotypes associated with the highest probabilities

$${P}_{m u t}^{max} ( S {T}_{i} ) ,$$

and also said probabilities.

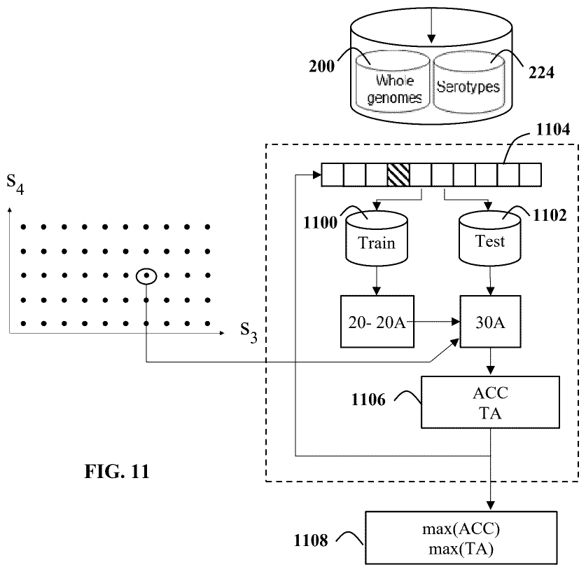

The values of the thresholds S₃ and S₄ are hyperparameters determined during step 220 of learning phase 30, preferably by means of cross-validation. With reference to FIG. 11, a grid of values for the thresholds S₃ and S₄ is first determined, and for each pair of thresholds in the grid, a determination of the correct degree of assignment and the average accuracy for serotype typing is performed by:
the partitioning of the whole genome database 200 and the serotypes 224 into a training database 1100 and a test database 1102 according to a cross-validation strategy 1104, for example a strategy called “ten-folds cross validation”;
performing the learning phase 20-201 on the training database 1102;
performing the prediction phase 30A for each genetic profile from the test database 1102 and calculating in 1106 the average accuracy ACC and the correct degree of assignment TA of the genetic profiles from the database 802 in the serotypes;
the selection, in 1108, of the maximum value of the average accuracies and correct degrees of assignment among the folds of the cross-validation strategy. These two values therefore correspond respectively to the average accuracy and the correct degree of assignment of the serotype prediction phase 30A for the thresholds S₃ and S₄. The pairs of thresholds having the best accuracy and degree are selected for serotype typing according to the invention. As a variant, the user can choose the accuracy and/or degree of assignment they desire, and the serotyping according to the invention selects the pair of thresholds whose accuracy and degree are closest to those chosen by the user.

In the variant just described, determining the serotype of a strain to be tested involves the calculations as described in relation to FIGS. 9 and 10. In another variant, for each taxon in the phylogenetic tree, the probability of each serotype in that taxon is calculated as a function of the serotype database 224. For example, the probability of a given serotype STⱼ in taxon Tᵢ is equal to the number of genomes grouped in this taxon that have this serotype divided by the total number of genomes in the taxon. These probabilities are stored in a reference table or dictionary, and the prediction of the serotype of a strain to be tested consists in first determining the taxon of the phylogenetic tree to which it belongs in the manner described in steps 308-312. Once the taxon has been identified, serotyping of the strain consists in querying the probabilities in the reference table associated with said taxon and providing the user with at least the serotype having the highest probability.

In the typing and serotyping variants just described, calculation of the values Pₘᵤₜ,

$${P}_{m u t}^{max} ,$$

and the assignment of the measured profile Pₘₑₛ are performed once the profile has been measured. As a variant, the already known profiles are directly associated with the taxon or the taxa of the binary phylogenetic tree, for example using a reference table. Specifically, the taxa are already known, the assignment calculation having been done beforehand. Notably, the profiles P_{obs} are assigned to their associated taxa by means of the calculations described previously. Thus, if the profile of a new strain is identical to a profile P_{obs}, the reference table is called up for this profile and the associated taxa are directly output. Similarly, when a new profile Pₘₑₛ is measured and differs from all the profiles in the reference table, its assignment is calculated as described previously, and this new profile is then stored with the taxon or taxa identified in the reference table. In this way, assignment calculations that have already been performed are avoided by simply calling up a reference table. As a variant, rather than a reference table, each already known profile is stored in a memory space dedicated to the taxon or taxa of the phylogenetic tree. In other words, the taxa in the tree are assigned “addresses” equal to the known and assigned profiles.

### D. Implementation Examples

D.1. Listeria monocytogenes

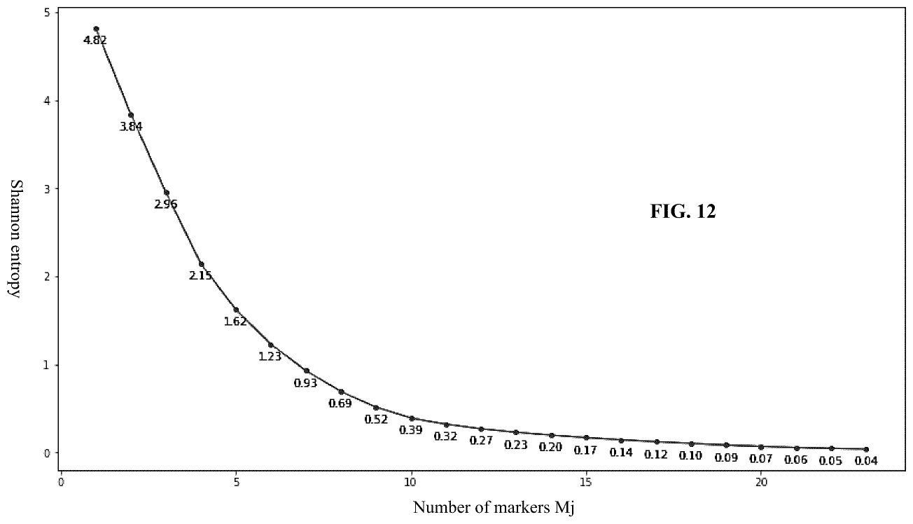

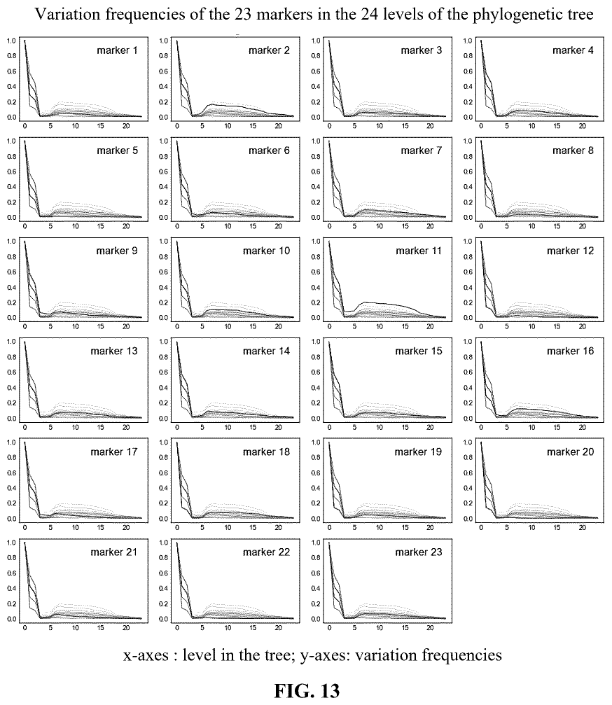

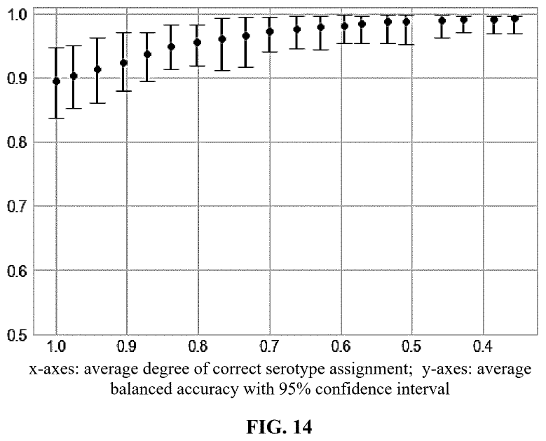

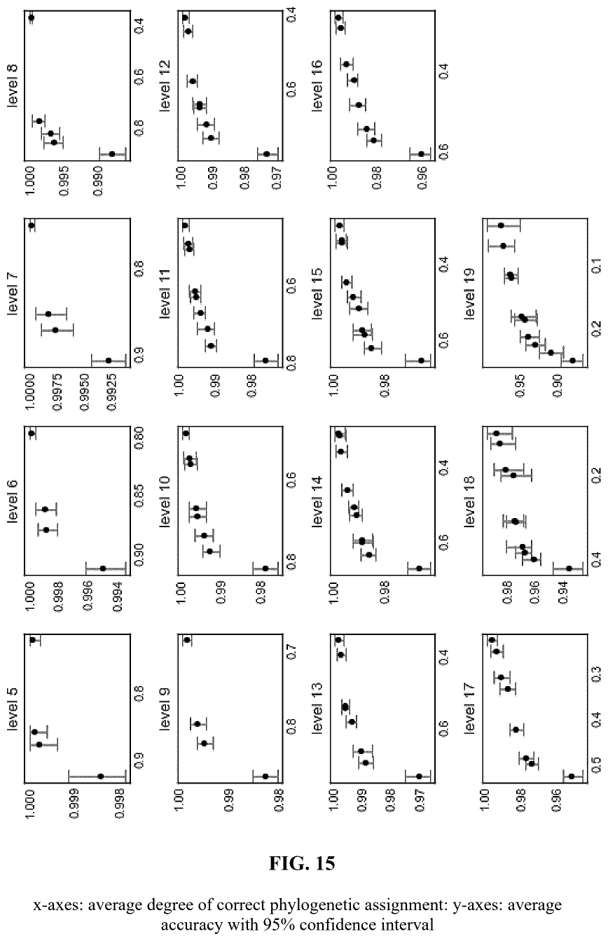

37 000 Listeria strains were collected, totally sequenced, and serotyped. The number of levels in the tree is equal to 23, which is the total number of targets on the Applicant's GENE-UP® PCR platform, and the distance decay law is selected for clustering so as to obtain a diversity similar to clonal diversity at a level between 10 and 15 in a phylogenetic tree including 24 levels. FIG. 12 illustrates the decrease in Shannon entropy of the genetic profile as markers are selected from the accessory genome at level 13 of the phylogenetic tree, with the entropy of the final profile only a few percent away from the free entropy at that level. FIG. 13 illustrates the frequencies of variation in the profile at different levels of the tree, with a curve in a graph corresponding to a taxon at that level and the bold curve corresponding to the frequency of the labeled marker. As may be seen in FIG. 14, which illustrates serotype typing among 12 known serotypes of Listeria, for a correct degree of serotype assignment of 100%, the average balanced accuracy is equal to 90%, demonstrating the efficacy of this typing according to the invention. It should be noted that it is possible to choose a higher accuracy for a satisfactory lower degree of assignment. Notably, for a degree of 80%, the accuracy is close to 95% with the 95% confidence interval contained entirely within the last decile. FIG. 15 illustrates the average accuracy of a level as a function of the correct phylogenetic degree of assignment. Once again, the excellent performance of the invention is noted, with these two values being greater than 95% and 70%, respectively, for all levels above level 15, and each close to 100% for levels above level 9.

### D.2. Salmonella

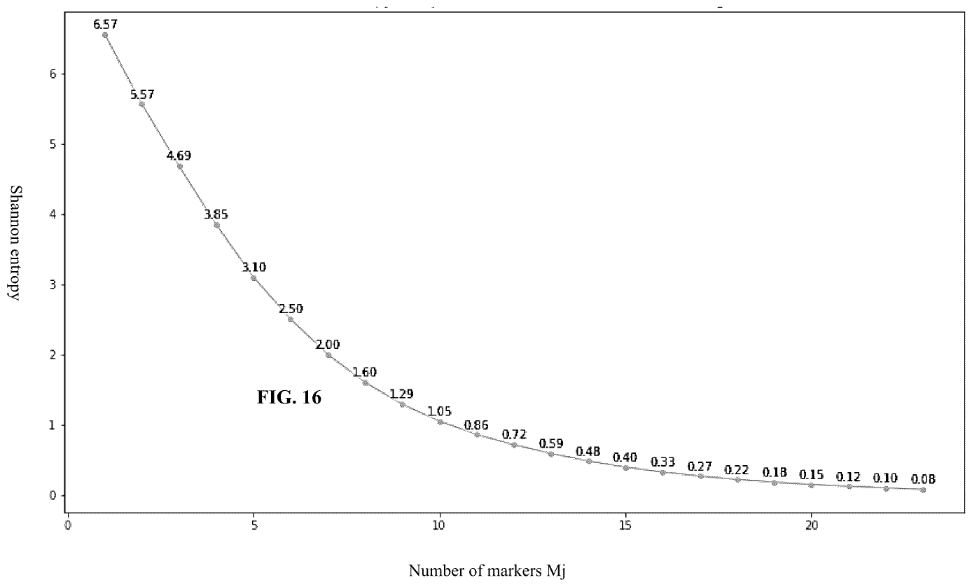

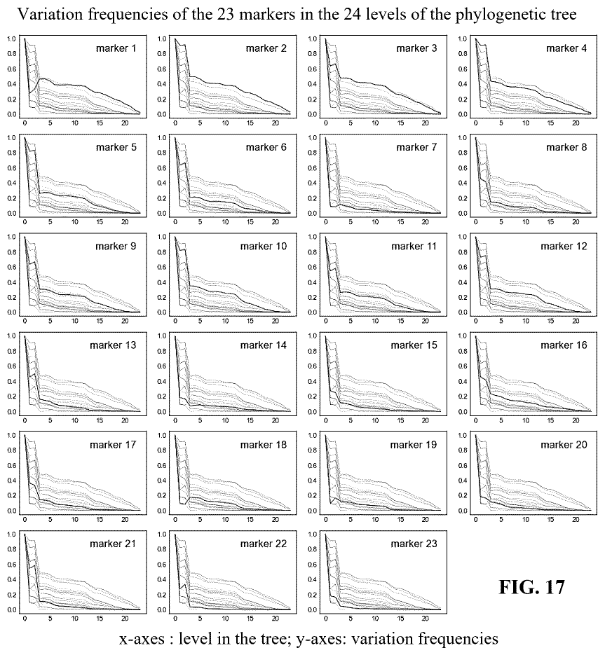

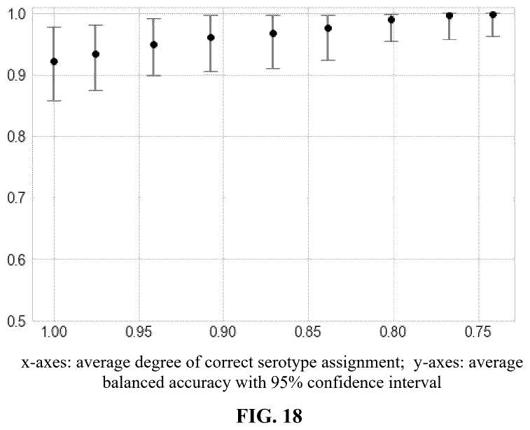

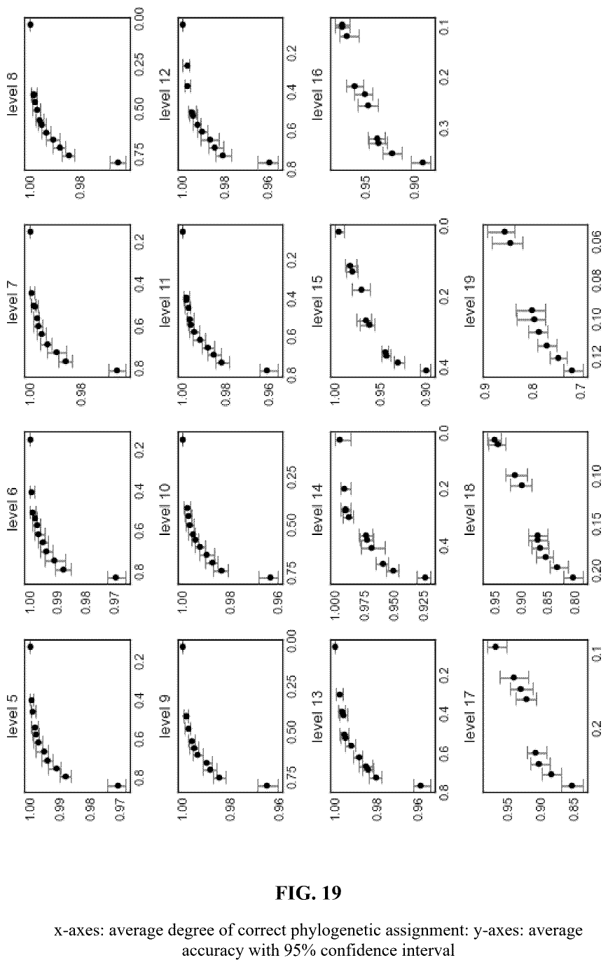

39 000 Salmonella strains were collected, totally sequenced, and serotyped. The number of levels in the tree is equal to 23, which is the total number of targets on the Applicant's GENE-UP® PCR platform, and the distance decay law is selected for clustering so as to obtain a diversity similar to clonal diversity at a level between 10 and 15 in a phylogenetic tree including 24 levels. FIG. 16 illustrates the decrease in Shannon entropy of the genetic profile as markers are selected from the accessory genome at level 13, with the entropy of the final profile being essentially zero. FIG. 17 illustrates the frequencies of variation in the profile at different levels of the tree, with a curve in a graph corresponding to a taxon at that level and the bold curve corresponding to the average frequency at that level. As may be seen in FIG. 18, which illustrates serotyping among the more than 2600 known serotypes of Salmonella, for a correct serotypic degree of assignment of 100%, the average balanced accuracy is greater than 90%, demonstrating the efficacy of this typing according to the invention. It should be noted that it is possible to choose a higher accuracy for a satisfactory lower degree of assignment. Notably, for a degree of 80%, the accuracy is close to 100% with the 95% confidence interval contained entirely within the last decile. FIG. 19 illustrates the average accuracy of a level as a function of the correct phylogenetic degree of assignment. Once again, the excellent performance of the invention is noted, with these two values being greater than 95% and 80%, respectively, for all levels above level 13.

### E. Preventive and Corrective Measures

The solution's ability to readily and quickly identify strains makes it possible to envisage its use for investigating root causes when contamination of a finished food product is identified during routine checks. This notably includes searching for the strain in the environment of the factory concerned, by means of surface samples collected with swabs, sponges or cloths, for example. The strain may also be searched for in the starting materials used in the creation of the finished product concerned, so as to link the contamination to one or more suppliers and/or transporters and/or storage locations. It may also make it possible to identify contamination during the laboratory detection phase, either by a laboratory strain or by their DNA, which may have skewed the release test that identified the presence of the species concerned. In this case, it would allow a plan to be drawn up to retest the finished product and avoid its destruction. Finally, the solution can be used during a health crisis so as to quickly characterize the strain(s) responsible and link them to strains identified in incriminated food, cosmetic, veterinary, or other products. Its usefulness therefore covers internal needs within a production facility, for example for food products, investigation needs during health crises or during regulatory audits on site. The use of this solution thus makes it possible to streamline and implement appropriate preventive and/or corrective measures for reducing or even eliminating the risk of recurrence of contaminations and, consequently, of infections.

### F. Computer Hardware Implementation of the Invention

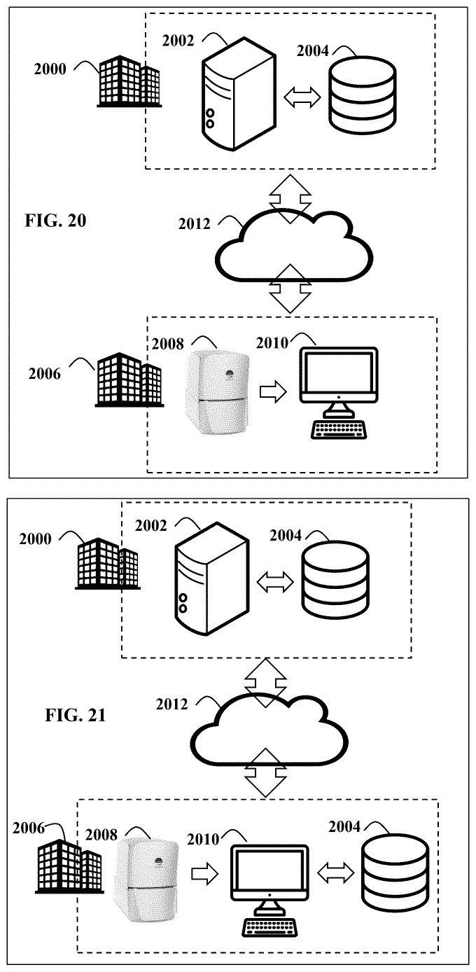

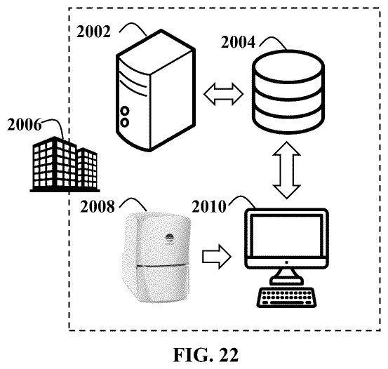

The learning phase 20, 20A and the prediction phase 30-30A, except for the steps of preparing the sample and measuring the genetic profile of the strains to be tested, are performed by computer, namely by means of hardware circuits comprising computer memories (cache, RAM, ROM, etc.) and one or more microprocessors or processors, organized or not in the form of computing nodes, necessary for executing computer instructions stored in the memories for performing said phases. Several architectures are possible, as illustrated, for example, in FIGS. 20-22. In a first architectural variant (FIG. 20), a first organization 2000, for example the Applicant, hosts or controls one or more computing servers 2002 associated with one or more databases 2002 for storing genomes, the phylogenetic tree, phylogenetic frequencies, observed genetic profiles, serotypes and serotype frequencies, and the learning phase 20, 20A is performed by the organization 2000 on its server(s) 2002. A second organization 2006, for example a microbiological laboratory, hosts a PCR platform 2008 connected to or incorporating a computer 2010 and performs part of the prediction phase 30, namely all the steps up to the measurement of the genetic profile of a bacterial strain to be tested. The measured profile is then sent via a remote connection network to the first organization 2000 for the implementation on the server 2002 of the rest of the prediction phase 30, 30A. The report produced is then sent via the network 2012 to the second organization 2006, which decides whether or not to take epidemiological measures on the basis of the report. A second architectural variant (FIG. 21) differs from the first in that a copy of the database 2004 and of the software performing the prediction phase 30, 30A is downloaded in the second organization, which uses the computer 2010 or a computing server (not represented) to perform all of the prediction phase 30, 30A. In a third variant (FIG. 22), all of the learning phases 20, 20A and prediction phases 30, 30A are performed by a single organization 2006.

### G. Extension of the Teaching of the Embodiment

An embodiment of the invention has been described using PCR to measure the genetic profile of a bacterial strain. As a variant, this profile is measured using a DNA chip targeting said markers in a manner known per se.

An embodiment has been described in which the complete genome database is independent, at least in its initial version, of the bacterial ecology of the organization performing the typing for its own needs. As a variant, this database consists essentially of bacterial strains originating solely from the organization. However, a minimal first version of the database can be provided to the organization and then supplemented with strains from the organization.

An embodiment applied to a bacterial species has been described. The invention also applies to yeasts and fungi.

A serotyping method has been described. Other subtypes of a species may be characterized, for instance their resistance and/or sensitivity to microbial agents or groups based on standard sequences.

A correct degree of assignment and also an accuracy, balanced or unbalanced, have been described as criteria for selecting the hyperparameters of the prediction. Other criteria are possible, for instance sensitivity and specificity or a confidence interval other than 95%.

1. An in vitro process for typing a microorganism contained in a sample, which involves:
d. providing computer storage means comprising:
a database of genetic profiles of microbial strains belonging to the microbial species of said microorganism, the profiles consisting of the presence or absence of a predefined set of genetic markers;
a phylogenetic tree of the species of the microorganism, assignments of the genetic profiles in the database to positions in the phylogenetic tree, frequencies of variation of the genetic profiles of the microbial strains in the database for predefined levels of the phylogenetic tree;
for at least part of the genetic profiles in the database, the membership of the genetic profiles to distinct microbial groups of the microbial species, the distinct groups being absent from the phylogenetic tree;
e. the measurement of the genetic profile of the microorganism;
f. computer-mediated determination of:
probabilities of variation of the genetic profiles belonging to the groups relative to the measured genetic profile, the probabilities of variation being calculated as a function of the frequencies of variation at a predefined level of the phylogenetic tree;
the microorganism's membership in one of the microbial groups if an identification performance criterion is met for the group, the criterion being met if at least one probability of variation for the group is greater than a first predefined threshold.

2. The process as claimed in claim 1, wherein:
the phylogenetic tree is constructed from a core genome of the microorganism species;
the predefined set of genetic markers is chosen from the accessory genome of the microorganism species.

3. The process as claimed in claim 1, wherein the number of markers of the genetic profiles is less than 50.

4. The process as claimed in claim 1, wherein:
the phylogenetic tree is constructed by the application of clustering based on genetic distances of a core genome of the microorganism species;
the genetic distances decrease from the root to the leaves of the tree, so as to define a level in the tree comprising between 150 and 350 differences in the core genome.

5. The process as claimed in claim 4, wherein:
the groups of microorganisms correspond to microbial serotypes of the microorganism species;
the phylogenetic tree comprises at least 10 levels;
the probabilities of variation of the genetic profiles belonging to the groups relative to the measured genetic profile are calculated as a function of the frequencies of variation of a level of the phylogenetic tree between 7 and 15.

6. The process as claimed in claim 1, wherein the markers of the genetic profiles are selected so as to minimize an entropy or impurity criterion at a level of the phylogenetic tree comprising between 150 and 350 differences in the core genome.

7. The process as claimed in claim 1, wherein the identification performance criterion also comprises the difference between the probability of variation for the group and the probabilities of variation for the other groups, the identification performance criterion being achieved if the difference is greater than a second predefined threshold.

8. The process as claimed in claim 7, wherein the identification performance indicator comprises a correct degree of assignment and/or a raw accuracy and/or a balanced accuracy.

9. The process as claimed in claim 1, which involves searching for root causes in the event of contamination of a food product.

10. The process as claimed in claim 1, wherein the measurement of the genetic profile of the microorganism is performed by means of amplification of target genetic sequences without performing complete sequencing.

11. A system for the in vitro typing of a microorganism contained in a sample, comprising:
d. computer storage means comprising:
a database of genetic profiles of microbial strains belonging to the microbial species of the microorganism, the profiles consisting of the presence or absence of a predefined set of genetic markers;
a phylogenetic tree of the species of the microorganism, assignments of the genetic profiles in the database to positions in the phylogenetic tree, frequencies of variation of the genetic profiles of the microbial strains in the database for predefined levels of the phylogenetic tree;
e. means for collecting and storing a measurement of the genetic profile of the microorganism;
f. computer-mediated means for determining:
probabilities of variation of the genetic profiles belonging to the groups relative to the measured genetic profile, the probabilities of variation being calculated as a function of the frequencies of variation at a predefined level of the phylogenetic tree;
the microorganism's membership in one of the microbial groups if an identification performance criterion is met for the group, the criterion being met if at least one probability of variation for said group is greater than a first predefined threshold.

12. The system as claimed in claim 11, configured to perform a process wherein the phylogenetic tree is constructed from a core genome of the microorganism species and the predefined set of genetic markers is chosen from the accessory genome of the microorganism species.

13. A computer program product recorded on a computer-readable support comprising instructions for executing step c. of a process as claimed in claim 1.

14. A composition for the in vitro typing of microorganisms contained in a sample by amplification of target nucleotide sequences, the composition comprising a set of oligonucleotides for the amplification of at least the set of genetic markers obtained as claimed in claim 6.

15. A process for preparing a biological sample comprising or liable to comprise a bacterial strain, for the purpose of typing the strain by a polymerization chain reaction, which involves placing the sample in contact with a composition as claimed in claim 14.

16. The process as claimed in claim 15, which involves lyzing the sample resulting from the placing in contact so as to release genetic material from the bacteria of the bacterial strain.

17. A process for preparing a sample which may comprise a microorganism, the process involving
the extraction of the genetic material included in the sample;
mixing of the extracted genetic material with a composition comprising a set of primers for amplification of a set of genetic markers obtained according to a process as claimed in claim 6.
